(19) United States
(12) Patent Application Publication
(10) Pub. No.: US 2009/0036382 A1
(43) Pub. Date: Feb. 5, 2009

(54) TGF-Beta Modulators and Use Thereof

(75) Inventors: Giorgio Bressan, Padova (IT); Paolo Bonaldo, Orsago (IT); Stefano Piccolo, Padova (IT); Giuseppe Lembo, Mugnano Del Cardinale (IT)

Correspondence Address: CLARK & ELBING LLP, 101 FEDERAL STREET, BOSTON, MA, 02110, US

(73) Assignees: Universita' Degli Studi Di Padova, Padova (IT); Universita' Degli Studi Di Roma "La Sapienza", Roma (IT)

(21) Appl. No.: 12/278,110

(22) PCT Filed: Feb. 1, 2007
(86) PCT No.: PCT/IB07/00233

§ 371 Date: Aug. 1, 2008

(30) Foreign Application Priority Data
Feb. 3, 2006 (IT) MI2006A000181

(51) Int. Cl. A61K 38/00; C07K 14/00; C12P 21/04; G01N 33/00; A61P 9/00

(52) U.S. Cl. 514/12; 530/324; 530/328; 530/327; 530/326; 530/325; 435/69.1; 436/86

(57) ABSTRACT

The present invention relates to molecules preferably of polypeptide nature with negative regulatory activity on the amount or activity of TGF-β through direct interaction with pro-TGF-β, and containing as active region a cysteine-rich polypeptide sequence defined as “EMI domain”, or its subfragments, wherein said “EMI domain” has at least 25% sequence homology to the ID NO2 sequence for pharmaceutical use. Even more preferably said polypeptide sequence consists of the EMI domain of the following proteins: emilin-1, emilin-2 and/or multimerin-2 or their subfragments having a length of at least 6 amino acids, capable of inhibiting the conversion of pro-TGFβ to mature TGFβ as anti-hypertensive drugs and polypeptides active on the cardiovascular system. The invention extends to the use of molecules which are known to negatively regulate TGF-β and to molecules which interfere with TGF-β binding to its receptors, or to inhibitors of TGF-β mRNA synthesis or TGF-β expression for the same therapeutic uses as anti-hypertensive drugs and polypeptides active on the cardiovascular system.

### FIELD OF THE INVENTION

The invention relates to antagonists of TGF-β activity as modulators of arterial hypertension.

### STATE OF THE ART

Arterial hypertension is an important risk factor for kidney, coronary and acute cerebral diseases. This condition is extremely widespread, since affects nearly one third of the adult population. A raise of arterial pressure can result from an increased function of the cardiac pump and/or an increased vascular resistance to the blood flow.

Recent studies have shown that also the elastic component of the extracellular matrix (ECM) has an important role in the mechanisms regulating arterial pressure (ref. D'Armiento J., 2003, J Clin Invest 112:1308-10). In particular, the study of transgenic mice lacking the elastin gene, wherein homozygous animals die as a result of arterial obstruction and heterozygous animals are stably hypertensive, contributed to elucidate the importance of ECM components of the vascular system in etiopathogenesis of hypertension (Li, D. Y., G. et al., 1998, J Clin Invest, 102:1783-7).

TGF-β is a very important growth factor in development and pathophysiology of blood vessels. It binds serine/treonine-kinase receptors thereby activating an intracellular signal. Mature TGF-β, i.e. active in receptor binding, is produced by a first proteolytic cleavage followed by an activation step releasing the active fragment from the precursor polypeptide chain.

However very little is known about the mechanisms that regulate the amount and the activity of TGF-β in vivo.

In fact, although so far TGF-β dysregulation has been associated mostly with tumors and fibrosis, it is well known that different TGF-β molecules (known TGF-β molecules include TGF-β1 TGF-β2 TGF-β3 see Goumans, M. J. et al., 2003, Trends Cardiovasc Med 13:301-7 are involved in several pathologies. For instance TGF-β has a dual role in tumors: in fact TGF-β acts by inhibiting proliferation of endothelial, epithelial and hematopoietic cells, but also by promoting tumor progression after an initial oncogenic event. Moreover, the immunomodulatory role of TGF-β is well recognized, as confirmed by the multifocal inflammatory pathology occurring in transgenic mice carrying the inactivation of at least one allele. On the other hand, the immunosuppressive activity measured in the microenvironment after TGF-β secretion may also lead to tumor promotion.

Several modulators of TGF-β activity are well known in the art: they act primarily by inhibiting the biological activity of TGF-β molecules including, for instance, antagonists of TGF-β or of the signal transduction pathway of TGF-β receptors. In clinical practice, modulators of TGF-β activity have been tested as antitumor or antimetastatic drugs or else as antifibrotic drugs, for instance in the therapy of pulmonary fibrosis. A review on therapeutic approaches based on TGF-β inhibition is presented in Yingling, J. M. et al., 2004, Nat Rev Drug Discov 3, 1011-1022.

So far, it has never been shown that TGF-β is directly involved in mechanisms regulating arterial pressure at the level of the extracellular matrix, as it has been found by the authors of the present invention, who have revealed the molecular mechanism underlying this regulation, thus opening the field to novel therapeutic approaches for treating hypertension by TGF-β inhibition or modulation of its circulating or local levels.

Nowadays, arterial hypertension is treated with vasodilating drugs with direct action (as for instance calcium antagonists, organic nitrates) or indirect action (as for instance inhibitors of the converting enzyme from angiotensin I to angiotensin II: the so called ACE-inhibitors, or β2-adrenergic receptor agonists), or by use of diuretics.

Each of these therapeutic approaches shows various side effects in hypertensive patients, also depending on possible concomitant administration of other interfering drugs.

Therefore, the need for new therapeutic approaches to treat hypertension is highly felt in the field, since it is well known (Staessen et al., 2003, Lancet 361, 1629-1641) that this condition represents a risk factor for fatal pathologies, such as coronaric infarction and thrombosis, and, if not treated, leads to renal failure.

### SUMMARY OF THE INVENTION

The present invention relates to molecules preferably of polypeptide nature characterized by a negative regulatory activity on the amount or activity of TGF-β through direct interaction with pro-TGF-β, and containing as active region a cysteine-rich polypeptide sequence defined as “EMI domain”, typical of the emilin protein family, or its subfragments or peptides derived from the EMI domain, wherein said “EMI domain” has at least 25% sequence homology to the ID NO 2 sequence for pharmaceutical use.

According to a particularly preferred aspect, said polypeptide sequence preferably consists of the EMI domain of the following proteins: emilin-1, emilin-2 and multimerin-2, corresponding respectively to sequences ID NO 2, 4, 6, 8, or their subfragments of at least 6 amino acids.

According to a preferred aspect, said molecules of polypeptidic nature, derived from or containing the EMI domain, have an anti-hypertensive activity and are active on the vascular system, in vascular remodeling, atherosclerosis, aneurysms and diabetic vasculopathies.

The invention is based on the identification of a novel regulatory mechanism of conversion of pro-TGF to mature TGF-β, regulated by emilins, and on the observation that alterations of said regulatory mechanism, especially those leading to increased amounts of mature TGF-β, result in hypertension. Therefore the invention extends to the use of molecules which are known to negatively regulate TGF-β where said negative regulatory activity interferes with conversion of pro TGF-β to mature TGF-β and acts as an inhibitor of the following classes of proteins with enzymatic activity: integrins αᵥβ6, extracellular matrix protease, including MMP-2 and MMP-9, plasmin, trombospondin-1 for use as anti-hypertensive agents and for their activity on the cardiovascular system, in vascular remodeling, atherosclerosis, aneurysms, diabetic vasculopathies.

Moreover, the invention extends to the use of TGF-β antagonists as anti-hypertensive agents, such as molecules which interfere with binding of TGF-β to its receptors, for instance anti-TGF and/or anti-pro-TGF antibodies or anti-TGF-β-receptor or to inhibitors of TGF-β mRNA synthesis and/or of TGF-β expression, as for instance siRNA specific for TGF-β or anti TGF-β antisense oligonucleotides, to silence the respective mRNAs thereby decreasing the expression and/or amount of mature and available TGF-β, or to molecules which inhibit kinase activity and signal transduction by TGF-β receptor.

According to a further aspect, the invention relates to a method employing EMI domains or their subfragments for selection of biologically active compounds, preferably with an activity regulating the conversion of pro TGF-β to mature TGF-β, potentially endowed with activity on the cardiovascular system, as anti-hypertensive agents.

According to a further aspect, the invention relates to the use of the EMI domain of emilins, or subfragments thereof in oncology.

### DESCRIPTION OF THE FIGURES

FIG. 1: Scheme of TGF-β1 processing.

FIG. 2. Panel A: HEK293T cells were transfected with a plasmid encoding the TGF-β type II receptor (tagged with the HA epitope) alone or along with a plasmid encoding the EMI-domain-GPI-anchor. Cells have been then treated with 125, TGF-β1 alone or together with excess cold TGF-β1, washed and treated with the crosslinker DSS (Disuccinimidyl suberate); at last, the cell extract was immunoprecipitated with anti-HA antibodies and proteins were separated by SDS-PAGE. Immunoprecipitations performed in presence of: control lane: ¹²⁵I TGF-β1; lane 2: ¹²⁵I TGF-β1+type II TGF-β1 receptor-HA tagged; lane 3: GPI anchored EMI domain+¹²⁵I TGF-β1+type II TGF-β1 receptor-HA tagged; lane 4: cold TGF-β+¹²⁵I TGF-β1+type II TGF-β1 receptor-HA tagged. Upper panel: Immunoprecipitation with anti-HA tag antibodies (αHA); lower panel western-blot analysis of the immunoprecipitate with anti-EMI GPI tagged antibodies (αEMI).

Panel B. Luciferase expression from the CAGA12-lux construct transfected in HEK293T cells alone (control bar) or in combination with a emilin1 expression vector (emilin 1 bar). After transfection, cells were treated (black bar) or not treated (white bar) with 5 ng/ml soluble recombinant TGF-β1 (R&D) (bars 1 and 2). Bars 3-6 relate to the treatment of cells co-transfected with emilin1 (or EMI-domain or emilin1ΔEMI) expression vectors alone (white bars) or along with proTGF-β1 coding vector (black bars). Values show the mean±st. dev.

Panel C. Luciferase expression from the CAGA12-lux construct transfected in HEK293T cells alone (control bar) or in combination with a emilin 2 or multimerin 2 expression vector (corresponding bars). Cells co-transfected with emilin 2 or multimerin 2 expression vector along with the expression vector for proTGF-β1. Values show mean±st. dev.

FIG. 3. Panel A and panel B: Activation of p15 promoter in MEF cells (+/+clear bars: cells isolated from wt mouse; −/− dark bars: cells isolated from emilin knock out mouse) after emilin 1 transfection concomitant or not with SB431542 treatment (panel A) or with SP600125 drug (panel B).

FIG. 4. Panel A: Immunoprecipitation with anti-Flag antibodies (ΔEMI) of extracts from HEK-293T cells transfected with a pro-TGF-β coding plasmid alone or in combination with a Flag-EMI-domain-GPI coding plasmid, and western-blot analysis of the immunoprecipitate with α-LAP (4A1) and 4A3) antibodies or αFlag antibodies (4A2).

Panel B: Immunoprecipitation and Western-blot analysis of extracts from HEK-293T cells transfected with a Flag-EMI-domain-GPI coding plasmid (lanes 1-4) and a pro-TGF-β coding plasmid (lane 2), as outlined in the scheme above the photograph. In lanes 3 and 4, LAP or the LAP+TGF-β complex preassembled in vitro was added to cell extracts prior to immunoprecipitation. Lane 1: the cell extract of samples transfected with the EMI-domain is directly immunoprecipitated with anti-LAP antibodies. The immunoprecipitate is then detected by Western-blot with anti-Flag antibody; lane 2: supernatants of samples transfected with the EMI-domain and with pro TGF-β were immunoprecipitated and blotted as described for lane 1. In lane 3, LAP was added to the sample prior to immunoprecipitation and in lane 4 the TGF β-LAP complex preassembled in vitro was added. In the lower part, a band present in cell extracts, detected by western-blot with anti-Flag antibody, shows comparable intensity in all lanes, thus proving that expression of the Flag-EMI domain-GPI construct is similar in all samples.

Panel C: Immunoprecipitation of endogenous emilin after assembly with proTGF-β produced upon transfection. HEK293 cells were transfected with proTGF-β and culture medium was immunoprecipitated with anti-LAP antibodies. The immunoprecipitate was detected by Western-blot with anti-Flag antibody, that recognizes proTGF-β, or with anti-emilin1 antibody. Lane 1: control of immunoprecipitation specificity, lacking anti-LAP antibody (−); lane 2: Immunoprecipitation with anti-LAP antibody.

Panel D: Western-blot of HEK293 cells co-transfected with plasmids coding pro-TGF-13 and emilin-1 or EMI domain. Cell extracts or supernatant were analyzed by western-blot.

Lane 1: transfection with pro-TGF-β alone; lane 2: transfection with pro-TGF-(3 and the EMI domain; lane 3: transfection with pro-TGF-β and emilin 1, as summarized in the scheme above the figure.

FIG. 5. Panel A: Western-blot analysis of supernatant from HEK293T cells transfected with pro TGF-β and emilin1. In a sample, cells were grown in presence of the furin-convertase inhibitor decanoyl-RVKR-CMK. Lane 1: transfection with pro TGF-β alone; lane 2: transfection with pro TGF-β and cell treatment with decanoil-RVKR-CMK; lane 3: transfection with pro TGF-β and emilin1.

Panel B: Western-blot analysis of supernatant from HEK293T cells transfected with a plasmid coding pro TGF-β1, furin-convertase SPC1 and EMI-domain, as indicated above the panel.

Panel C: Western-blot analysis of supernatant from Mouse embryonic fibroblasts (from +/+mice in lane 1, and from −/− mice in lanes 2 and 3) transfected with a plasmid encoding Flag-pro TGF-β and E. coli β-Galactosidase, that was further incubated with the furin-convertase inhibitor RVKR-CMK (lane 3). The western-blot was developed with anti-Flag antibodies (α-FLAG, upper panel), anti-LAP antibodies (αLAP, middle panel) and with a control antibody (α βGal, lower panel).

FIG. 6. Panel A: Cell-mixing experiments with HEK293T cells (see explanation in the text, Example 6). The graph shows the activation of reporter gene (luciferase) expression induced by TGF-β secreted by cells transfected with a TGF-β plasmid. The following abbreviations are used in the figure: R: HEK293T, Responder cells (transfected with the plasmid carrying luciferase under control of the CAGA12 promoter, induced by TGF-β); S: HEK93T, Stimulator cells (transfected with the plasmid driving the expression of pro-TGF-β). In the cases shown, R and S cells were cotransfected, in addition, with the plasmid encoding emilin-1 (+E). Mock, cells transfected with the CMV-lacZ control plasmid alone.

Panel B: Cell-mixing experiments with Mouse embryonic Fibroblasts (MEF), under conditions similar to those described in panel A (former FIG. 3L). The graph shows the activation of reporter gene (luciferase) expression under control of the p15 promoter induced by the TGF-β secreted by MEF cells stimulated with SP600125. The following abbreviations are used in the figure:

wt: MEF from normal or wild type mice; wt^{R}: MEF from normal R mice, Responder (transfected with p15); wt^{S}: MEF from normal S mice, Stimulating, treated with SP600125.

ko: MEF from emilin 1 knock-out mice; ko^{R}: MEF from emilin 1 knock-out mice, R, Responder, transfected with p15; ko^{S}: MEF from emilin 1 knock-out mice, S, Stimulating, treated with SP600125.

Column bars 1 and 2 relate to a control cell-mixing experiment wherein MEF wt^{R} were mixed with untreated MEF wt or ko and luciferase levels are comparable and basal. The other column bars refer to cells that have been mixed, as indicated under each column.

Panel C: HEK293T cells transfected with the plasmids encoding the TGF-β1 precursor (proTGF-1), emilin-1 or emilin-1-KDEL (which is not secreted) along with the CAGA12-lux reporter construct and with CMV-lacZ (enzymatic dosage of lacZ enables normalization of reporter gene expression levels in the different samples). Inset: comparison of emilin 1 and emilin 1-KDEL expression in samples in lanes 3 and 4, by Western-blot with anti-emilin 1 antibodies.

### DETAILED DESCRIPTION OF THE INVENTION

### Definitions

By TGF-β it is meant the Transforming Growth Factor beta. TGF-β is composed by two subunits of 12 KD linked by disulphide bridges. By TGF-β it is meant TGF-β1, 2, 3. TGF-β1 is particularly preferred in the present invention. The sequences of these growth factors are well known for several animal species. The GenBank accession No. for the human sequences correspond to: TGF-β₁ and TGF-1 precursor: NP_{—}000651 (protein) and NM_{—}000660 (cDNA), TGF-β2 and TGF-β2 precursor: NP_{—}003229 (protein) and NM_{—}003238 (cDNA) and TGF-β3 and TGF-β3 precursor: NP-003230 (protein) and NM_{—}003239 (cDNA).

ProTGF-β TGF-β is not synthetized by the cells as such, but as precursor molecule of about 50 KD termed proTGF-β. Said precursor is cut by furin-type proteases into two parts, the propeptide, termed LAP (Latency Associated Peptide), and TGF-β. Once pro TGF-β proteolysis has occurred, the two resulting polypeptides remain associated in a complex termed LAP/TGF-βeta or SLC (Small Latent Complex). TGF-β in this complex cannot interact with its receptor and is therefore inactive. The dissociation of TGF-β from SLC is defined as TGF-β activation, because, after this event, TGF-β is active in that it is able to interact with its receptor.

LAP: It is the propeptide moiety which is released proteolytically from the proTGF-β precursor, as defined above.

LTBP: Latent TGF-β Binding Proteins constitute a group of four proteins with structural homology. Three of them (LTBP-1, -3 and -4) are covalently bound to proTGF-β by disulphide bridges inside the cell and secreted in this form by the cell. The LTBP-proTGF-β or LTBP-SLC complex is named LLC (Large Latent Complex). For a review on these aspects of TGF-β regulation, see Annes, J. P. et al., 2003, J Cell Sci 116, 217-224.

Emilins: The emilin family comprises proteins carrying a EMI domain, composed of a cysteine-rich region of about 80 aa at the NH2 end, an alpha-helical region in the middle portion and a region homologous to the C1q globular domain (gC1q domain) at the carboxy-terminal end, as described by Braghetta et al., 2004, Matrix Biol 22.

### DETAILED DESCRIPTION OF THE INVENTION

The present invention is based on the discovery that emilin family proteins (an acronym for Elastin Microfibrils Interface Located proteIN), endowed with the EMI domain, a cysteine-rich region of approximately 80 amino acids, and participating to the elastic component of the extracellular matrix, are extremely important for regulation of arterial pressure and in the pathogenesis of hypertension. They affect vascular remodeling and resistance by modulating local TGF-β availability.

In fact, it is well known that emilins belong to a protein family characterized by a unique structural arrangement comprising, from the N-terminus: a signal peptide, the above mentioned EMI domain, an alpha helical region in the middle portion and a region homologous to the C1q globular domain (gC1q domain) in the carboxy-terminus (see the nomenclature agreed by the experts at the site http://www.gene.ucl.ac.uk/nomenclature/genefamily/#.html#HGNC_table2).

Based on structural homologies, particularly at the level of the EMI region, to date the emilin family includes: emilin-1, the first to be isolated (Bressan et al., 1993, J Cell Biol 121, 201-212; Doliana et al., 2000, J Biol Chem 275, 785-792), emilin-2 (Doliana et al., 2001, J Biol Chem 276, 12003-12011), multimerin 1 (Braghetta et al., 2004, Matrix Biol 22, 549-556; Hayward et al., 1995, J Biol Chem 270, 18246-18251), which is a protein secreted by endothelial cells and platelets, multimerin 2 (Braghetta et al., 2004; Christian et al., 2001 J Biol Chem 276, 48588-48595; Leimeister, C. et al., 2002, Dev Biol 249, 204-218), which is also produced by endothelial cells.

The functional characterization of each component of this family has not been completed yet, although recent data have shown that antibodies against emilin 1 inhibit the deposition of elastin by aortic smooth muscle cells in vitro (Bressan et al., 1993, already cited).

Even though transgenic mice carrying an inactivation of the emilin 1 locus appear normal, since homozygotes are fertile and have an apparently normal life cycle, a more subtle investigation shows structural alterations of elastic fibers and of cellular morphology within arteries (Zanetti, M. et al, 2004, Mol Cell Biol 24, 638-650). The physiological role of these molecules has not been clarified yet, however the authors of the present application have found that the TGF-β precursor is a target molecule and have identified target organs and tissues for this protein family.

In fact, the subject of the present invention, in all its preferred and derived aspects, originates from the extraordinary observation that transgenic mice carrying at least one mutated, inactivated or silenced allele of an emilin family gene, compared to wild type, and in which emilin expression is detectably and stably lower than normal, are phenotypically hypertensive.

The authors of the present invention have demonstrated, on this basis, that emilin family proteins regulate the level of mature (available) TGF-β in vessel extracellular matrix by binding to pro-TGF and that the amount of mature TGF has a direct effect on arterial pressure. In particular, a reduced level of emilin proteins in the elastic component of the extracellular matrix results in increased levels of mature TGF-β (available), hypertension and alterations of the vascular system.

The TGF-β superfamily comprises TGF-β1, TGF-β2 and TGF-β3, as defined above. In addition to binding the same receptor, these molecules share very similar processing, high structural similarity and a very high amino acid sequence homology which exceeds 70% in the portion corresponding to the mature protein.

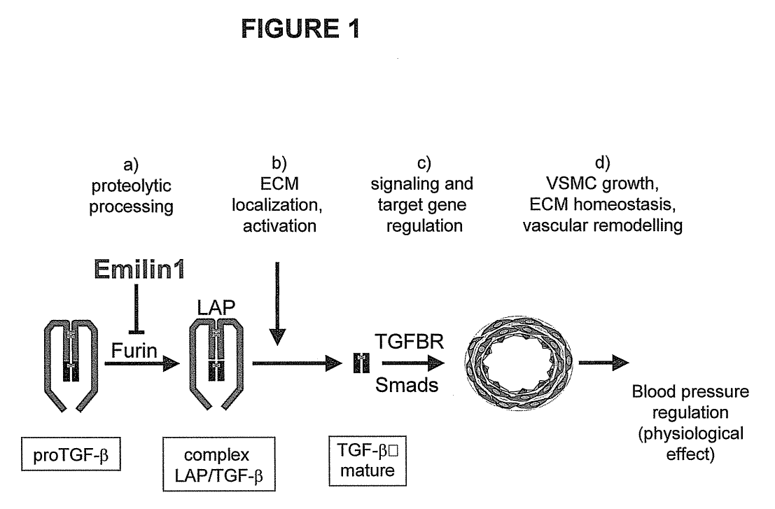

As mentioned in the introduction, TGF-β is not synthetized by cells as such, but as proTGF-β. Pro-TGF is a dimer of a 390 amino acid-precursor stabilized by disulphide bridges. Glycosylation of the dimer (about 80 KD) increases the molecular weight of circulating pro-TGF (dimeric and glycosylated) to 90-100KD. ProTGF-β is cut by furin-type proteases into two parts, the propeptide, termed LAP (Latency Associated Peptide), and TGF-β, which remain associated in a complex termed LAP/TGF-βeta or SLC (Small Latent Complex). TGF-β is inactive in this complex. The dissociation of TGF-β from SLC is defined as TGF-β “activation”, because, after this event, TGF-β is able to interact with its receptor. It is believed that the “activation” releasing mature TGF-β is performed by trombospondin or proteases or specific integrins or low pH (Annes J-P. et al., 2003, J Cell Sci 116, 217-224). A scheme of the whole TGF-β processing is presented in FIG. 1.

Mature TGF-β binds a serine/threonine kinase receptor (TGFBRII), which transduces the signal by recruiting other intracellular TGF-β receptors, phosphorylating the type I receptor and then receptor regulated Smads, which in turn control, at transcriptional level, the expression of other genes or sets of genes.

The authors of the present invention have found that emilin family proteins, through their association with immature TGF-β precursor, inhibit its conversion to the mature protein by inhibiting the proteolytic cleavage thereby the relative amount of mature TGF-β. This regulatory mechanism turns out to be especially important in the Extra-Cellular Matrix of vessels, where the modulation of TGF-β production, and especially a higher availability of mature TGF-β, results in an increase of arterial pressure. In fact, transgenic mice carrying an inactivation of the emilin coding gene due to gene targeting, as for instance the Emilin-1 gene knock out mouse already described by Zanetti M. et al. Mol. Cell. Biology, 2004 24:638-650, have an apparently normal phenotype but, upon more careful analysis, turn out to be phenotypically hypertensive due to the above identified mechanism.

Therefore, the inventors have identified a new control mechanism of arterial pressure and of cardiovascular alterations, which may even only predispose to a raise of arterial pressure. Moreover, they have identified the physiological function of emilin proteins, preferably of emilin 1, emilin 2, multimerin 1 and multimerin 2 as negative regulators of the processing of pro-TFG to mature TGF-β. Furthermore, in this context, a novel in vivo function of TGF-β has been identified.

They have clarified that the modulation of the amount of mature TGF-β at the ECM level, and therefore the interactions between at least one emilin, especially between their EMI domain and pro-TGF-β, are directly involved in the regulation of arterial pressure. In fact, the cause of hypertension discovered in mice carrying the inactivation of the emilin locus (emilin 1 −/−) is to be ascribed to altered peripheral resistance, due to a reduction of the diameter of the aorta and of all the vascular system, even though the arteries of −/− mice, although of smaller caliber, show a normal elastic response in presence of physiological pressure levels. Moreover, emilin −/− mice show a different proliferative capacity of smooth muscle cells.

The coordinated role of emilins, especially of emilin1 and TGF-β, in the regulation of blood pressure has been further shown in vivo by a gene interaction experiment whereby emilin1 knockout mice were crossed with mice genetically deficient for TGF-β1: reduced TGF-β1 dosage due inactivation of one allele restores normal pressure levels.

Therefore, it follows that the use of TGF-β antagonists or of inhibitors of the proteolytic conversion from immature precursor to the mature form enable regression of the hypertensive phenotype, by decreasing the amount of available TGF-β.

Proteolytic cleavage of TGF-β precursor is performed physiologically by furin-convertase-type enzymes, which have the property to cleave the peptide bond in COOH-terminal position relative to two paired basic residues, as for instance K-R and R-R. Therefore any molecule which, like emilins, binds the cleavage site recognized by proprotein-convertases in the TGF-β molecule, and prevents its processing, is usable to inhibit the hypertensive mechanism.

In fact, it has been shown that the interaction between TGF-β precursor and at least one protein of the emilin family, or at least the EMI region of one of them, regulates physiologically, in a negative manner, the amount of TGF-β produced.

Therefore, following the experimental observations, the present invention relates to emilin proteins, especially emilin-1, emilin-2 and multimerin2, or their subfragments, such as the EMI domains, that are functionally capable of binding the TGF-β precursor blocking its proteolytic cleavage to mature TGF-β, for pharmaceutical use in order to modulate arterial hypertension through their binding to pro TGF-β.

For the purpose of the present invention, said molecules are defined as TGF-β antagonist able to decrease the amount of mature TGF-β available by reducing, in this specific case, the conversion of pro TGF-β to TGF-β at the extracellular level.

Therefore, according to a first embodiment, the invention relates to isolated and preferably recombinant human emilins, especially emilin 1, emilin 2, multimerin 1, multimerin 2 (whose amino acid sequences are known in the GenBank with accession No. NP_{—}008977, NP-114437 e NP_{—}079032, respectively) as TGF-β modulators.

In particular, the invention relates to molecules comprising a cysteine-rich polypeptide sequence defined as “EMI domain”, or its subfragments, wherein said “EMI domain” has at least 25% sequence homology to the highlighted sequence in SEQ ID NO 2, for pharmaceutical use. EMI domain sequences are identified and highlighted in the enclosed Sequence Listing and correspond to the following fragments of the respective emilin proteins, preferably of human origin:
emilin-1: fragment corresponding to residues 55-131 of the human protein with sequence deposited in the GenBank with accession No. NP_{—}008977 (gene sequence: GenBank GeneID 11117), corresponding to sequences ID NO 1 and ID NO 2 (DNA and protein);
emilin-2 (also known as Basilin): fragment corresponding to residues 43-120 of the human protein with sequence deposited in the GenBank with accession No. NP-114437 (gene sequence: GenBank GeneID 84034), corresponding to sequences ID NO 3 and 4 (DNA and protein);
multimerin-1: fragment corresponding to residues 206-283 of the protein: GenBank acc. No. NP_{—}031377; (gene sequence: GenBank GeneID 22915), corresponding to sequences ID NO 7 and 8 (DNA and protein);
multimerin-2 (previously designated EndoGlyx-1 or emilin-3): fragment corresponding to residues 54-131 of the protein: GenBank acc. No. NP_{—}079032; (gene sequence: GenBank GeneID 79812), corresponding to sequences ID NO 7 and 8 (DNA and protein);

Particularly preferred for use as antihypertensive agents or for preparation of antihypertensive drugs, or of drugs active on the cardiovascular system, are the EMI domains or EMI domain-derived peptides from emilin-1, emilin-2, multimerin 1 and multimerin 2, preferably the EMI domains with sequence ID NO 2, ID NO 4, ID NO 6, ID NO 8 or sequences derived from said domains (carrying, for instance, a deletion of few amino acids) and the chimeric proteins comprising said domains.

It is part of the present invention the use of said molecules for the preparation of antihypertensive drugs, and/or drugs active on the vascular system and/or drugs active on vascular remodeling and/or atherosclerosis, and/or aneurysms and/or diabetic vasculopathies. The effect of said molecules can be extended to the induction of structural alterations in the extracellular matrix as well as to vascular remodelling, increased lumen diameter, increased thickness of the media layer of the vessel wall, if possible even inducing a higher response of smooth muscle cells to contractile stimuli.

According to the findings from the authors of the present invention, the cysteine-rich domain, termed “EMI domain”, is functionally capable of inhibiting the proteolytic conversion of proTGF-β to mature TGF-β, thereby exerting, for the purpose of the present invention, the same effects as the whole protein from which it is derived.

The invention comprises polypeptides having an amino acid homology higher than 25%, or more preferably an homology of at least 28%-30%, of 40%, of 50%, of 60%, of 70%, of 80% and of 90% or higher than 90%, comprising all intermediate homology values, to the EMI domain of human emilin-1, taken as reference (GenBank GeneID 22915 and seq ID NO:2) or its fragments or derivatives. Said domains have the same inhibitory function on conversion of pro TGF-β to mature TGF-β as their respective whole protein.

The subfragments derived from the amino acid sequence of the EMI region, which retain the activity according to the present invention, have preferably a length shorter than 50 amino acids and longer than 6 amino acids, even more preferably comprised between 7 and 30 or even more preferably between 8 and 20 or even more preferably comprised between 8 and 15 amino acids.

Through the same regulatory mechanism of pro TGF-β processing, the medical use of said molecules, and of polypeptides according to the invention, extends to their use as immunomodulators or in the field of oncology, where TGF-β plays a fundamental role reviewed by Yingling, J. M. et al., 2004, Nat Rev Drug Discov 3, 1011-1022.

Said peptides and/or polypeptides are preferably modified so that they are stabilized against the action of proteases as they contain one or more amino acid in D-form.

Moreover, they can contain, as an alternative, modified and/or uncommon and/or non-natural amino acids, such as for instance: 2-aminoadipic acid, 3-aminoadipic acido, b-alanine, 2-aminobutyric acid, 6-aminocaproic acid, 2-aminoheptanoic acid, 2-aminoisobutyric acid, 3-aminoisobutyric acid, desmosin, 2,2′ diaminopimelic acid, 2,3′ diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, 6-N-methyl-lysine, N-methylvaline, norvaline, norleucine, ornithin. Moreover, the polypeptides according to the invention may comprise sequences or biochemical modifications which do not change the biological activity as defined in the invention, but enhance stability or change product compartmentalization or localization. As an example, modifications such as myristoylation, amidation, glycosylation, GPI anchoring, integrin anchoring via RGD sequence can be considered. The peptides/polypeptides of the invention are conveniently produced by chemical synthesis or in recombinant form, which is produced by inserting the nucleotide sequence encoding said functionally active domains in suitable expression vectors and then in recombinant organisms, preferably prokaryotes or lower eukaryotes such as yeast, from which they can be purified in large amount. Specific nucleotide sequences encoding said domains are enclosed in the sequence listing. However, degenerate sequences according to the genetic code, encoding a EMI domain with at least 25% homology to the EMI domain of emilin 1 having SEQ ID NO 2, can also be used for realization of the method. These are preferably the EMI domains of emilin2, multimerin-1 and multimerin-2, respectively corresponding to sequences ID NO 4, 6, 8, and their subfragments interacting with pro-TGF-β.

Alternately, polypeptides and peptides of the invention are obtained by enzymatic or chemical cleavage from chimeric or fusion proteins, both recombinant and of extractive origin, or they are the chimeric or fusion proteins themselves.

Therefore, it is part of the invention the use of nucleotide sequences encoding emilins and other EMI domains as defined above, as well as their derived subfragments having a length of at least 6 aminoacids, in order to obtain, by recombinant methods, the proteinaceous active substances for preparation of drugs preferably for therapy and/or prevention of hypertension, of drugs active on the vascular system and in the field of oncology. Said molecules are also active in vascular remodeling, atherosclerosis, aneurysms and in diabetic vasculopathies, as well as studying the etiopathogenesis of hypertension, vascular remodeling, atherosclerosis, aneurysms and diabetic vasculopathies.

According to this embodiment of the invention, vectors comprising the above defined nucleotide sequences are part of the invention: said vectors are preferably expression vectors and may comprise both sense and antisense sequences for expression of emilins and/or multimerins.

The compounds identified by the authors of the present invention are also used for research and selection of lead compounds with antihypertensive activity, of compounds active on the vascular system, in vascular remodeling, atherosclerosis, aneurysms and in diabetic vasculopathies. Said compounds are also useful to enhance the response of vessel smooth muscle cells to contractile stimuli or, for instance, for the screening of libraries of chemical, biochemical and biological compounds with an activity mimicking emilins or their EMI domains and subfragments, for instance in competition assays detecting the amount of mature TGF-β produced from protein precursor(s), also in the field of oncology. According to a preferred embodiment, said assays detect the competing activity of compounds on the proteolytic activity converting pro TGF-β to mature TGF-β and/or LAP/TGF-β complex.

Therefore, in addition to the use of the above mentioned peptides or peptide-mimetics, the present finding opens the way, as it appears to the skilled man, to totally innovative therapeutic approaches aimed at reducing, by different mechanisms, the level of mature or active TGF-β in the vessel extracellular matrix.

Thus, the invention extends to the use of already known TGF-β inhibitors acting by the mechanisms listed below, without being limited thereto, having the final effect to decrease or antagonize the activity of TGF-β, in some instances already known as antitumor or antifibrotic agents, for use as antihypertensive agents. Well known molecules are for instance:
Inhibitors of the conversion of TGF-β precursor to mature TGF-β. This category includes both the inhibitors of proteolytic enzymes involved in the first phase of TGF precursor processing to LAP/TGF-β complex, by enzymatic processing of TGF-β precursor (pro TGF-β) to LAP/TGF-β, and the enzymes responsible for activation and release of mature TGF-β from the LLC complex and/or LAP/TGF-β (Large Latent Complex). The proteolytic enzymes involved in the first phase are preferably proprotein convertases, or even more preferably the subtilisin-like proprotein convertases (SPC), especially furin-convertase. Furin inhibitors are for instance the dec-RVKR-CMK peptide or polyarginine sequences, comprising at least 3 arginines covalently bound by peptide bonds, more preferably comprising 4-9 arginine residues (for a review see Fugere M & Day R., 2005, Trends Pharmacolol Sci, 26: 294-301), as for instance L-hexa-arginine, both in L- and D-configuration.

The second phase of LAP/TGF-β complex “activation” is dependent on extracellular matrix proteases MMP-2 and MMP-9 (MMP: Matrix Metal Proteases), plasmin, trombospondin (TSP-1), or also integrins, e.g. αvβ6; inhibitors of these proteases are well known in the art (Sluijter, J. P. et al., 2005, Vascular remodeling and protease inhibition-bench to bedside. Cardiovasc Res. (online pub. Dec. 28, 2005).
Molecules interfering with binding of TGF-β to its receptors, such as antagonists of mature TGF-β and/or of its receptor, like TGF-β antagonists (according to the definition accepted in pharmacological field—capable of binding the receptor thereby interfering with signal transduction or signaling induced by the natural ligand), as for instance soluble forms of TGF-β receptor (which binds and sequesters circulating TGF-(3 preferably from the ECM compartment), or antibodies neutralizing TGF-β/TGFBRII binding.

TGF-β neutralizing antibodies, such as 1D11 which neutralizes TGF-β1, 2, 3 (Ruzek et al., 2003, Immunopharmacol Immunotoxicol 25, 235-257), or antibodies such as for instance lerdelimumab (Cordeiro M., 2003, Immunopharmacol Immunotoxicol 25, 235-257) or metelimumab (Bayes et al., 2005, Methods Find Exp Clin Pharmacol 27, 193-219) or, in addition, the antibody GC-1008 (Yingling et al., 2004, already cited) are well known in the field of TGF-β antagonists and proved to have limited side effects. Once it is known the amino acid sequence of the variable antibody fragment or optionally the nucleotide sequence, derivatives of said antibodies can be obtained in recombinant form by suitable genetic manipulations.

The preparation of soluble receptor forms, able to interfere with TGF-β binding to its receptors, optionally conjugated to carrier proteins, prepared for instance according to genetic engineering techniques, is well known in the art: soluble receptor forms have been designed also for other growth factors and/or cytokines (e.g. TNF-α) and are well known also for type II TGF-β receptor in a form conjugated to the immunoglobulin Fc region, as described in Yang Y. et al., 2002, J Clin Invest 109: 1607-1615.

Inhibitors of TGF-β mRNA synthesis and/or of TGF-β expression, as for instance siRNA specific for TGF-β or anti TGF-β antisense nucleic acids, which can be easily designed from the known TGF-β sequence and suitably modified, using for instance phosphorothioate nucleotide synthesis; oligonucleotides specific for TGF-β are already well known with the abbreviations AP-11014 (TGF-1) and AP-12009 (TGF-β2), and they are described in (Yingling et al., 2004, already cited).

Short sequences of TGF-β1-3 interfering RNA are used according to the invention in order to silence their respective mRNAs, thereby reducing the expression and thus the amount of available TGF-β. Said antisense nucleic acid and oligonucleotide sequences can be obtained by well known methods (for instance described in Soutschek et al., 2004, Nature 432, 173-178);
Inhibitors of TGF-β receptor kinase activity, as for instance derivatives or variants of one or more pharmacophores which bind the receptor kinase domain, as for instance classes identified with the abbreviations LY550410, LY 580276, SB 505124, SD-208 (Byfield, S. D., and Roberts, A. B., 2004, Trends Cell Biol 14, 107-111; Sawyer J. S. et al., 2004, Bioorg Med Chem Lett 14, 3581-3584). Inhibitors of receptor kinase activity are preferably compounds that compete with ATP or comprising a hydrogen bond acceptor group, wherein said acceptor group is preferably a 4-fluorophenyl group, chinoline, pyrazol 2 substituted with naphthyridine, imidazopyridine, pyrazolopyridine;
Positive modulators of emilin expression, as for instance transcriptional activators (for instance transcription factors which are already produced in solubile and/or recombinant form, such as for instance SP1).

The above described negative modulators of TGF-β expression, as for amount or signaling (generally defined as TGF-β antagonists for the purpose of the present invention) or TGF-β antagonists known in the literature, or of their derivatives having the same biological activity, and furthermore inhibitors of the conversion of TGF-β precursor to mature TGF-β, and their derivatives or obvious variants, and inhibitors of TGF-β transcription and/or translation and their derivatives or obvious variants, inhibitors of TGF-β receptor kinase activity, and their derivatives or obvious variants, positive modulators of emilin expression, and their derivatives or obvious variants, are in the present application claimed for use for preparation of drugs for therapy and/or prevention of hypertension, for preparation of drugs active on the (cardio)vascular system, of drugs active in vascular remodeling, on atherosclerosis, aneurysms, diabetic vasculopathies, as well as for use in studies on ethiopathogenesis of hypertension, vascular remodeling, atherosclerosis, aneurysms and diabetic vasculopathies, wherein said activities may be accompanied by vascular remodeling and/or increase of vessel lumen and/or of the thickness of the media layer of the vessel wall and/or by enhanced response of vessel smooth muscle cells to contractile stimuli.

Therefore, the present invention comprises all the molecules that block or interfere with the activity of TGF-β by the above listed mechanisms, without being limited thereto, negatively modulating its biological activity, especially at the level of the extracellular matrix.

The various classes of molecules which preferably but not exclusively act by the above identified mechanisms are of chemical, proteinaceous, amino acid, nucleotide nature and, as seen above, share a negative modulatory activity on the activity of mature TGF-β and are therefore defined as global antagonists. Said molecules comprise variants or derivatives that can be obtained according to methods well known in the art and are identified in the present invention for each class.

According to an especially advantageous aspect of the present invention, TGF-0 processing inhibitors, emilin agonists or TGF-β antagonists, exert their effects preferably at a local level. In fact, even the physiological inhibition of TGF-β processing is performed outside the cell, in the extracellular matrix compartment, as shown in detail in the experimental part.

Therefore, the present invention extends to pharmaceutical compositions wherein active principles according to the invention are combined with suitable excipients and/or diluents or with other active principles for systemic or local administration. Moreover, active principles according to the invention are preferably delivered to their site of action, the extracellular matrix, by drug-delivery systems as for instance liposomes, lipid nanoparticles and/or bio-delivery systems and/or molecular “targeting” systems mediated by protein sequences (for instance viral or “RGD” domains).

According to a further embodiment, the invention relates to a therapeutic method comprising the administration of molecules according to the invention, individually or in combination with other drugs, to a patient affected by hypertension, diabetic complications or atherosclerosis.

### EXPERIMENTAL PART

### Methods

For standard molecular biology, biochemical and immunoassay methods (e.g. construction of plasmids used in the present invention) one refers to the manual “Current Protocols in Cell Biology” Howard R. Petty Wayne State University, Detroit, Mich.; Juan S. Bonifacino, Mary Dasso, Joe B. Harford, Jennifer Lippincott-Schwartz, and Kenneth M. Yamada (eds.), Copyright © 2003 John Wiley & Sons, Inc. The manual is currently termed “Current Protocols”.

### Materials

The construct for emilin 1 expression was engineered in pCS2 vector (Rupp R. A. et al., 1994, Genes Dev 8, 1311-1323) (for a physical map of the vector, see the site faculty.washington.edu/rtmoon/pcs2+.html) as described (Zanetti M. et al., 2004, Mol Cell Biol 24, 638-650); the vector used for proTGF-β expression in MEF and HEK293T cells is pCDNA3.1 (Invitrogen). Vectors containing the coding sequence for human pro TGF-β are described in Young and Murphy-Ullrich, 2004 (Young and Murphy-Ullrich, 2004, J Biol Chem 279, 38032-38039). The human pro TGF-β cDNA sequence is reported in the data bank (NP_{—}000660; protein 000651).
The vector for Furin/SPC 1 expression is pCDNA3.1 (Invitrogen)
p15 and CAGA 12 promoter fragments are described in Jonk L. J. et al., 1998, J Biol Chem 273, 21145-21152 ed in Li J. M. et al., 1995, J Biol Chem 270, 26750-26753.
Constructs containing Flag sequences are described in Young G. D and Murphy-Ullrich J E, 2004, J Biol Chem 279, 38032-38039.
Constructs containing the Flag epitope (e.g. Flag-EMI-domain-GPI) were prepared as described in Chubet R G et al. Biotechniques 1996; 20(1):136-141 and detected by anti-Flag antibody (Kodak Inc.).
Constructs containing the GPI anchor were prepared as described in the Manual “Current Protocols in Cell Biology” Howard R. Petty Wayne State University, Detroit, Mich.; Juan S. Bonifacino, Mary Dasso, Joe B. Harford, Jennifer Lippincott-Schwartz, and Kenneth M. Yamada (eds.), Copyright C 2003 John Wiley & Sons, Inc. (GPI-anchor). In the construct Flag-EMI domain-GPI, the Flag sequence was inserted at the N-terminus after the signal peptide, while the GPI-anchor sequence was placed at the COOH-terminal end.
The construct emilin 1-KDEL was obtained by inserting the KDEL coding sequence described by Munro S., and Pelham, H. R., 1987, Cell 48, 899-907 upstream the emilin 1 STOP codon.
The construct containing the type II TGF-β receptor with HA epitope was described in Wrana J. L. et al., 1994, Nature 370, 341-347.
supplier of the drugs used: SP600125 (Calbiochem), SB431542 (Tocris).
Purified recombinant LAP and TGF-β1 proteins were purchased from (R&D Systems)

Suppliers: anti-hemoagglutinin antibodies (Sigma), anti-LAP (R&D Systems), anti-Flag (Sigma), anti-emilin Bressan G. et al., 1993, J Cell Biol 121, 201-212.

### Example 1

### Culture of Cells Isolated from Emilin-2 and Multimerin-2 Transgenic Knock-Out and from Wild Type Animals

The preparation of mice carrying the inactivation of a locus encoding a EDEN group protein (or emilins) by gene targeting was performed according to standard techniques. Basic vectors are well known in the art: their features are described for instance by Mansour, S. L, et al., 1988, Nature 336, 348-52 and Kaestner, K. H. et al., 1994, Gene 148, 67-70.

In particular, the preparation of emilin 1 knock out animals is described by Zanetti, M. et al, 2004, Mol Cell Biol 24, 638-50. Transgenic mice carrying an inactivation of the emilin 1 locus appear normal, homozygotes are fertile and have an apparently normal life cycle; however, a more subtle investigation shows that they have structural alterations of elastic fibers and of cellular morphology within arteries and, surprisingly, they are hypertensive. The hypertensive phenotype is also found in emilin 2 and multimerin 2 knock out animals and in mice belonging to strain C57Bl/6O1aHsd (Harland, Ind.-USA) which is homozygous for a spontaneous deletion encompassing 365 Kb between positions 60,976 and 61,431 Mb of chromosome 6, comprising six exons of the Snca gene and eight exons of the multimerin1 gene (Specht, C. G., and Schoepfer, R., 2004, Genomics 83, 1176-8). Because they lack the entire transcribed portion of the multimerin-1 gene, these mice represent a natural knock-out of the complete multimerin-1 gene.

The conclusions from experiments carried out with transgenic animals carrying an inactivation of emilin 1, emilin 2 or multimerin 2 locus by gene targeting and with mice carrying a natural inactivation of the multimerin 1 locus, due to spontaneous mutation, are that hypertension is caused by increased peripheral resistance. The vascular phenotype of said animals is similar: a reduction of diameter is found in the aorta and in all the vascular system, however the arteries of −/− mice, although with smaller caliber, show a normal elastic response at physiological pressure levels.

The preparation of cell cultures from −/− and w.t. mice, for instance primary fibroblasts, MEF and smooth muscle cells, was carried out as described in Hogan, B. et al., 1994, “Manipulating the mouse embryo. A laboratory manual.” Cold Spring Harbor Laboratory Press, Cold Spring Harbor, N.Y. or in Freshney I. “Culture of animal cells: A Manual of Basic Technique” ed. 4th, 1991, John Wiley & Sons, Inc.

The interruption or incompleteness of the locus encoding EDEN family proteins, emilin 1, 2, multimerin 1 and multimerin 2, was ascertained by Southern-blot analysis performed on tissues from knock out o null mice, as described in Zanetti M. et al., 2004, Mol Cell Biol 24, 638-650:

### Example 2

### Characterization of the Mechanism of Interaction Between TGF-β and Emilin

Experiments were performed according to standard methods (Massague, 1987). Briefly, a solution of iodinated TGF-β1 (Amersham) was applied to HEK293T cells grown in 24 well plates and transfected with a plasmid carrying the sequence of TGFBRII (type II TGF-β receptor) fused to the HA epitope. After appropriate incubation and washing, the cell layer was exposed to a solution of DSS (disuccinimidyl suberate), a compound that favors formation of covalent cross-links between interacting molecules. The cell layer was extracted with immunoprecipitation buffer (RIPA-buffer) and subjected to the immunoprecipitation procedure with anti-HA antibody. The immunoprecipitate was solubilized with FSB (final sample buffer), separated by SDS-PAGE and radioactive complexes were detected by autoradiography. Molar excess of unlabeled TGF-β was used in parallel experiments. In other experiments, cells were simultaneously transfected with TGFBRII and with expression vectors driving expression of the emilin-1 EMI domain bound to GPI (glycosylphosphatidylinositol), a moiety which anchors the EMI domain to the cell membrane, that was tagged with the Flag epitope (Flag-EMI-domain-GPI).

Transfected cells, usually HEK293T, were incubated in Optimem (Invitrogen) for 24 hours prior to collection. Cell harvesting was with buffer containing 25 mM TRIS pH 7.5, 150 mM NaCl, 2.5 mM EDTA, 10% glycerol, 1% NP40 and protease inhibitors (Roche) maintained at 4° C. The cell suspension was shaken in order to disaggregate cells and cell residues were removed by centrifugation at 4° C. The so obtained cell lysate was diluted with 4 volumes of washing buffer (50 mM TRIS pH 7.5, 150 mM NaCl, 2.5 mM EDTA, 10% glycerol, 1% NP40, 0.5 mM MgCl2, protease inhibitors and 0.2% BSA). Antibodies were then added to the diluted lysate and samples were incubated overnight at 4° C. with gentle shaking. Protein A Agarose (10 μl) was then added and the incubation was continued for 2 hours. Beads were collected by centrifugation, washed three times with washing buffer without BSA and containing 0.3% NP40 and proteins were removed from Agarose in FSB for SDS-PAGE.

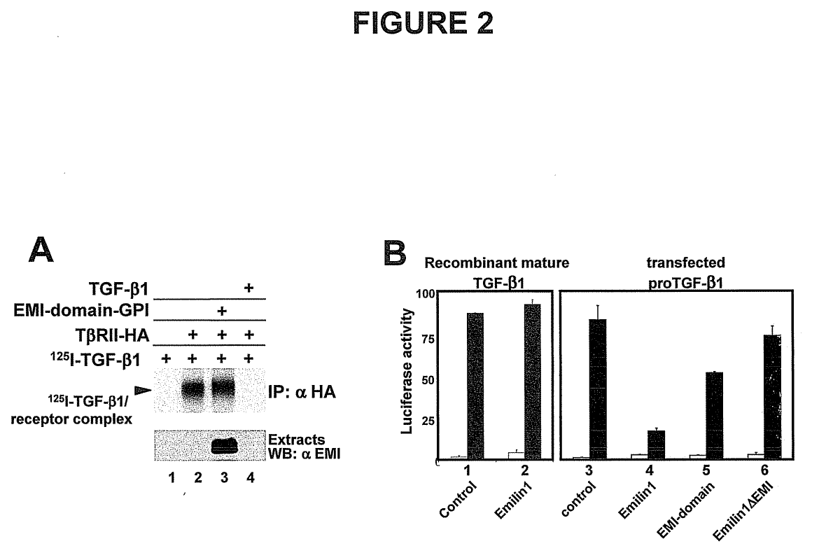

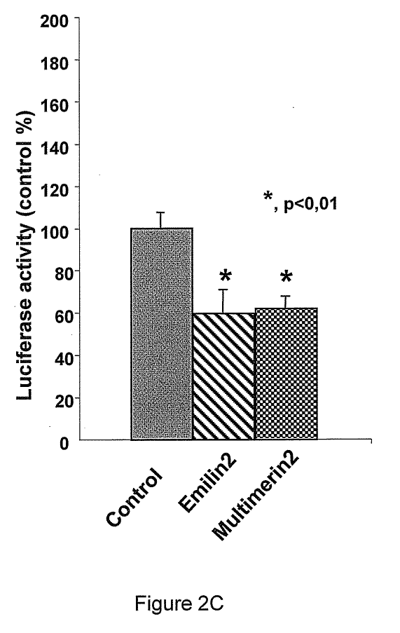

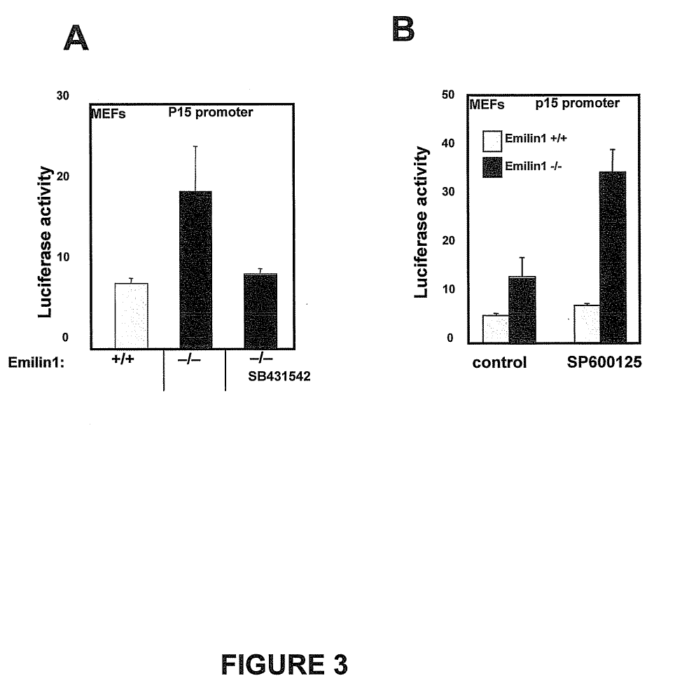

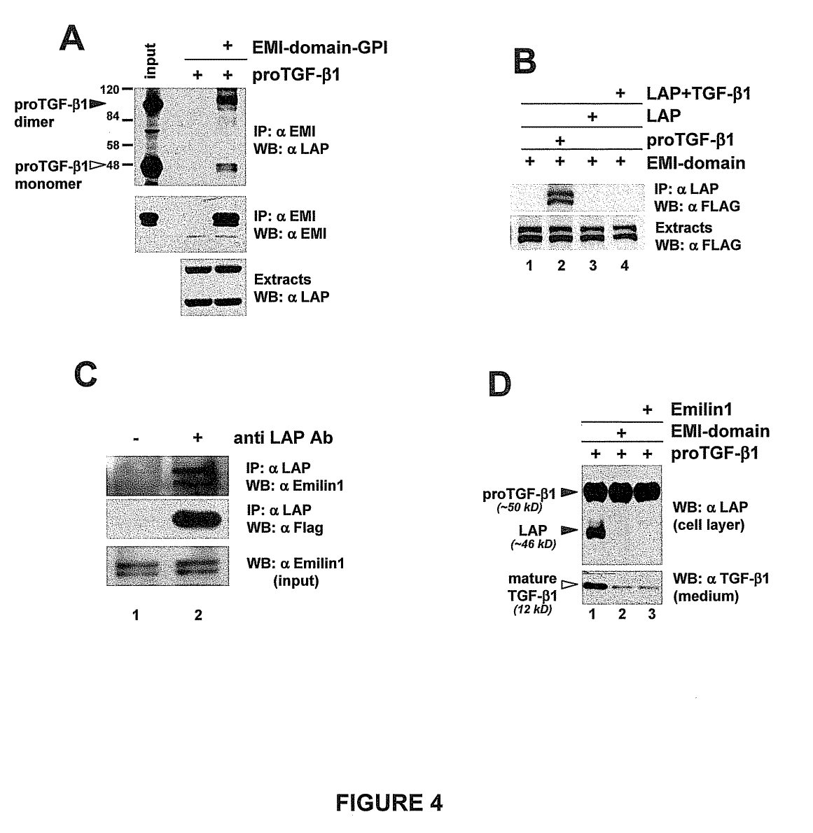

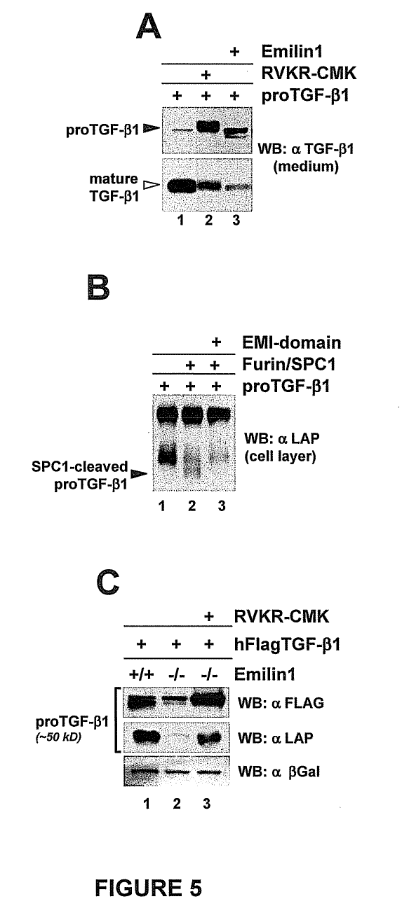

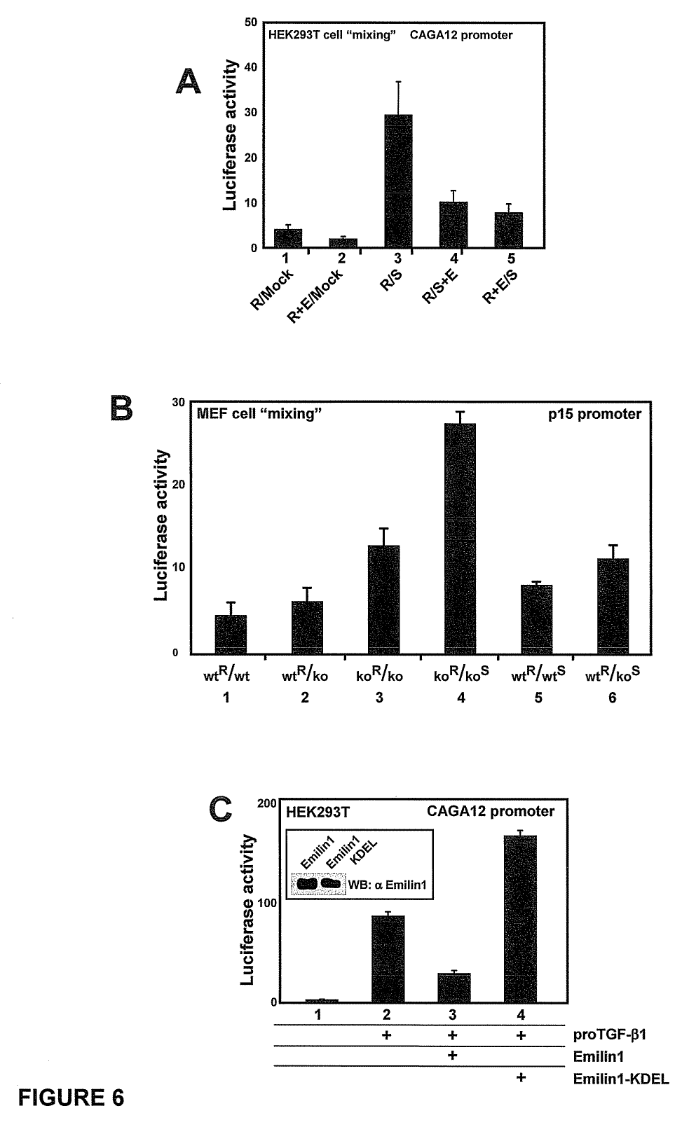

As shown in FIG. 2A (summarizing different treatments and/or transfections with a series of +above the figure), cells transfected with TGFBRII alone and exposed only to iodinated TGF-β show an electrophoretic band corresponding to the covalent TGFBRII/TGF-β complex (upper panel, lane 2). This band disappears with the addition of excess unlabelled TGF-β (lane 4), but not when the EMI domain is expressed (lane 3). Expression of the EMI domain was validated by western blotting with anti-Flag antibodies (lower panel). The data show that the EMI domain does non compete with TGF-β for binding to TGFBRII. Therefore, emilin-1 inhibition of TGF-β signaling does not affect its interaction with the receptor.

To verify how emilin or the EMI domain can contrast TGF-β1 activity independently from receptor binding, HEK293T cells were transfected with a construct coding luciferase placed under control of a TGF-β activated promoter: CAGA12-lux (Jonk et al., 1998) alone or in combination with a emilin1 expression vector (500 ng) as shown in FIG. 2B. After transfection, cells were treated (black bar) or not treated (white bar) with 5 ng/ml recombinant TGF-1 (lanes 1 and 2). Column bars from 3 to 6 report luciferase expression values in experiments with cells co-transfected with the expression vector for emilin1 (or EMI-domain or emilin1ΔEMI) alone (white bars) or in combination with the vector encoding pro TGF-β1 (black bars). Values show the mean±st. dev. Thus emilin (or the EMI domain) does not antagonize mature TGF-β, but inhibits pro TGF-β. As control, the emilin protein lacking the EMI-domain was used, which showed no effect on pro TGF-β (last column).

A comparable activity was found for emilin 2 and multimerin 2 (FIG. 2 panel C), indicating that emilin 2 and multimerin 2, like Emilin 1, antagonize TGF-β activity at the of pro-TGF-β level.

### Example 3

### Fibroblasts Isolated from Emilin ko Mice Produce Higher Amounts of Active TGF-β Compared to those Isolated from Wild Type Mice

MEF primary cultures were transfected with plasmid p15-lux (p15-luciferase) (Li et al., 1995) driving transcription of the luciferase marker gene under control of the p15^{INK4B} gene promoter. Since the p15 promoter is activated by TGF-β (i.e. p15 is a TGF-β target gene), the presence of the growth factor induces higher luciferase levels, which can be measured with a luminometer.

As seen in FIGS. 3A and 3B, transcriptional activation is higher in −/− mutant cells compared to control cells. Moreover, following treatment of cells with the drug SB431542 (Inman et al., 2002), which inhibits receptor response to TGF-β, a similar response is measured in normal and mutant cells (3A), whereas addition of the drug SP600125, which increases transcription of endogenous TGF-β as result of JNK activation (Ventura et al., 2004), greatly increases the response in emilin1 −/− cells (3B).

Therefore, the higher response of the p15-lux construct in MEF cells derived from emilin1 −/− animals suggests that mutant cells produce higher amounts of active TGF-β compared to wild type cells.

### Example 4

### Study on Emilin-1 Interaction with TGF-β Precursor

### Pro TGF-β

To verify whether TGF-β or its precursor is the emilin target, HEK293T cells were transfected with a plasmid encoding pro TGF-β1 alone or with an expression plasmid for Flag-EMI domain-GPI. Cell extracts were prepared and subjected to the immunoprecipitation procedure with anti-Flag antibodies, as described in example 2. Immunoprecipitates were subjected to SDS-PAGE and Western blotting (WB) with anti-LAP antibodies.

The results shown in FIG. 4A show that pro TGF-β (detected by WB with anti-LAP antibodies) is part of the complex immunoprecipitated with anti-Flag-EMI domain-GPI antibodies (lane 3); Flag-EMI domain-GPI (middle panel), detected by Flag-specific antibodies, is part of the same complex. Pro TGF-β does not enter the complex if Flag-EMI domain-GPI is omitted from the transfection (lane 2), although its presence in the cell extract can be demonstrated with anti-LAP antibodies (lane 2). Therefore, it is possible to conclude that pro TGF-β interacts specifically with the EMI-domain of emilin-1.

To better investigate the type of interaction existing between pro TGF-β and emilin, and to map the region involved in the TGF-β precursor molecule, HEK293T cells were transfected with plasmid Flag-EMI-domain-GPI. Results are shown in FIG. 4B. Pro TGF-β encoding plasmid was cotransfected in addition as indicated with + in the summary scheme above the figure. The cell extract was then subjected to immunoprecipitation with anti-LAP antibody and to Western blotting with anti-Flag antibody after different treatments, as indicated on the side or above the photograph. More specifically: lane 1 and lane 2: samples were directly immunoprecipitated; lane 3: LAP was added to the sample prior to immunoprecipitation; lane 4: the TGF-β/LAP complex, assembled in vitro starting with TGF-β and LAP, was added to the sample prior to immunoprecipitation.

As shown in FIG. 4B, the western-blot turns out to be positive only in lane 2, where pro TGF-β and EMI-domain are simultaneously present, thus the EMI-domain interacts only with the intact pro TGF-β molecule, and not with the LAP/TGF-β complex (also known as SLC: small latent complex) or only with LAP.

In the lower inset of panel 4B, a control western blot done with anti-FLAG antibodies detecting the EMI domain construct showed that expression of said construct was comparable in different experiments.

To ascertain that TGF-β interaction with emilin1 also occurs under physiological conditions of emilin 1 expression, HEK293T cells were transfected with the pro TGF-β encoding plasmid and culture medium was subjected to immunoprecipitation with anti-LAP antibody followed by western blotting with anti-LAP or anti-emilin1 (lower inset). Results are presented in FIG. 4C, showing that TGF-β co-precipitates also endogenous emilin1 (not overexpressed as result of transfection) physiologically produced by HEK293T cells. Therefore, it can be concluded that the interaction of pro TGF-β with emilin1 occurs also in presence of physiological levels of emilin1 expression.

To verify whether emilin or its EMI domain inhibit TGF-β or its precursor, HEK293T cells were cotransfected with plasmids driving the expression of pro TGF-β1 (fused to the FLAG-domain, even though the antibody used were directed at LAP and TGF-β proteins) and emilin-1 (or EMI-domain). FIG. 4D shows cell extracts (upper inset) and supernatants (lower inset) analyzed by western-blot with anti-LAP antibodies or anti-TGF-β1 antibodies, respectively. As seen in lane 3, the presence of emilin1, or of the EMI-domain (lane 2) decreases the intensity of LAP and TGF-β1 bands. Therefore both emilin-1 and EMI-domain inhibit the conversion of pro TGF-β1 (about 50 kD) to LAP (about 46 kD) and mature TGF-β1 (about 12 kD). The anti-Flag antibody used was obtained from Kodak Inc; for the preparation of Flag constructs, see Younjg and Murphy-Ullrich (Young and Murphy-Ullrich, 2004).

### Example 5

### Comparison of the Effects of Emilin-1 and Furin Inhibitors on Conversion of Pro TGF-β to Mature TGF-β

As reported in the summary scheme above FIG. 5A, HEK293T cells were transfected with the plasmid encoding pro TGF-β1 (lane 1) and cotransfected with emilin-1 (lane 3) or treated with decanoyl-RVKR-CMK peptide (Decanoyl-Arg-Val-Lys-Arg-chloromethylketone, Alexis Corporation, Lausen, Switzerland) (lane 2) at a concentration of 100 μM. The peptide is an irreversible and competitive inhibitor of furin proprotein convertase and of other SPC family members (Proprotein Convertase); in addition, it inhibits pro-MMP2 dependent furin activation. The supernatant was then loaded on SDS-PAGE and detected by Western-blot with anti-TGF-β1 antibody. As seen in FIG. 5A, comparing the relative amount TGF-β precursor and mature TGF-β in lane 1 with those in lanes 2 and 3 (detected with anti-TGF-β1 antibody), the intensity of the band corresponding to mature TGF-β1 decreases both in presence of cotransfected emilin-1 (lane 3) and of the inhibiting peptide, as shown in lane 2. An increase of the precursor band in lanes 2 and 3 is inversely proportional to the decrease of TGF-β. Therefore it can be concluded that this assay detects an effect of emilin-1 on TGF-β1 processing that is similar to the effect of Furin inhibitors.

To further investigate the role of emilin-1 on TGF-β processing, HEK293T cells were transfected with plasmids encoding pro TGF-β1, Furin/SPC 1 and EMI-domain, as shown in the scheme above FIG. 5B. Cell extracts were loaded on SDS-PAGE and detected by Western-blot with anti-LAP antibody. As seen in the figure, Furin transfection induces the band corresponding to the product of proteolytic cleavage of pro TGF-1 (lane 2), while the simultaneous presence of the EMI-domain blocks the effect of Furin (lane 3). Therefore this experiment shows that emilin-1 inhibits proTGF-β cleavage by Furin.

In a subsequent experiment, Mouse Embryo Fibroblasts (MEF) derived from ko (−/−) or wild type (+/+) mice were transfected with a plasmid encoding Flag-proTGF-β1; the Furin inhibitor decanoyl-RVKR-CMK was added to the indicated samples. The culture medium was then subjected to western blotting with anti-Flag (FIG. 5C, upper inset) or anti-LAP (middle inset) antibody. Results show the presence of much lower pro TGF-β levels in the supernatant of −/− cells compared to w.t. cells. Addition of the Furin inhibitor results in increased pro TGF-β1 levels, suggesting that emilin 1 or its EMI domain protect pro TGF-β from proteolytic degradation. To control the expression levels, western blot (in the lower inset) with an anti-β gal antibody was developed instead.

Therefore, the physiological role of emilin1 is to protect pro TGF-β from the proteolytic action of Furins, and emilin1 is required to prevent proTGF-β processing.

### Example 6

### Determination of the Cellular Compartment where Emilin Inhibition of Pro TGF-β Processing Occurs

To determine where emilin and pro TGF-β interact (whether intracellularly or extracellularly), two types of HEK293T cells, responder (R) and stimulator (S), were prepared by transfecting the CAGA12-lux plasmid in the former and of the proTGF-β coding plasmid in the latter cells. Therefore, R cells respond to TGF-β by activating the luciferase reporter gene, while S cells produce TGF-β and can stimulate the former cells only if TGF-β is secreted. To determine where emilin-1 blocks TGF-β processing, R or S cells were cotransfected with the emilin 1 coding plasmid, and this transfection is indicated with the initial +E under the corresponding column bar in FIG. 6A.

To be able to compare the results obtained under the various conditions, all cells were cotransfected also with the CMV-lacZ vector. Mock refers to cells transfected with CMV-lacZ plasmid alone. The experiment shows that mixing of R and S cells induces activation of CAGA12-lux (compare lanes 1 and 3). Emilin 1 inhibits activation of the reporter construct not only when it is present in S cells (lane 4) but also when it is produced only by R cells (lane 5), and in the latter case exerts its effect on TGF-β processing only after secretion. Therefore, the inhibition of pro TGF-β processing by emilin 1 occurs in the extracellular compartment.

This conclusion was also confirmed under more physiological conditions, using MEF cells (primary cultures of mouse embryonic fibroblasts) for transfection of R(esponders) or S(timulator) constructs. Results are shown in FIG. 6B. The experimental conditions are similar to those described in FIG. 6A, however pro TGF-β production by MEF primary cultures is stimulated with SP600125 administered to S cells prior to mixing with R cells. The drug SP600125 induces transcription of pro TGF-β by inhibiting JNK (Ventura et al., 2004). Moreover, unlike the previous experiment, R cells were transfected with p15-lux carrying luciferase under control of the p15 promoter. The following abbreviations are used:

wt: MEF from normal or wild type mice; wtR: MEF from normal R mice, Responder (transfected with p15); wtS: MEF from normal S mice, Stimulator, treated with SP600125.

ko: MEF from emilin 1 knock-out mice; ko^{R}: MEF from emilin 1 knock-out mice, R, Responder, transfected with p15); ko^{S}: MEF from emilin 1 knock-out mice, S, Stimulator, treated with SP600125.

Results in FIG. 6B show that the combination wtR with untreated cells (wt or ko) leads to comparable expression of the reporter gene (column bars 1 and 2). In line with the experiments shown in FIG. 3B, expression levels increase with the combination koR/ko (column bar 3). When mixing R and S cells, the ko^{R}/ko^{S} combination results in higher activation of luciferase expression (compare column 4 with column 5). Surprisingly, the wt^{R}/wt^{S} combination results in expression levels similar to the wt^{R}/ko^{S} combination (compare columns 5 and 6); the low inhibition obtained in this case can be explained only by assuming that S cells secreted intact (uncleaved) pro TGF-β and that emilin1 secreted by R cells further protected pro TGF-β from furin proteolysis.

Therefore, also in MEF cells, hence in more physiological conditions, inhibition of pro TGF-β processing by emilin1 occurs in the extracellular environment.

As further validation of the above observations, HEK293T cells were cotransfected with expression plasmids carrying the cDNAs shown in the summary scheme above the figure, along with CAGA12-lux and CMV-lacZ constructs, and luciferase expression levels were measured.

Results are shown in FIG. 6C, where it can be seen that emilin1 inhibits the stimulating action of transfected pro TGF-β (compare column bars 2 and 3). Retention of emilin1 in intracellular organelles (ER or Golgi apparatus), determined by the presence of KDEL sequence in the construct used for transfection (Martire et al., 1996, J Biol Chem 271, 3541-3547), abolishes its inhibitory action on the effects of pro TGF-β (column bar 4). Therefore emilin1 must be secreted in order to inhibit pro TGF-β processing.

### CONCLUSIONS

Considering the overlapping phenotypes of mice deficient for the various emilins (emilin-1, emilin-2, multimerin-1 and multimerin-2) and the results described above, it can be concluded that the interaction with emilins (or with their corresponding EMI domains) in the extracellular space protects pro TGF-β from proteolytic processing and that said interaction leads to the inefficient release of biologically active TGF-β. Said interaction is not limited to TGF-β1, as inferred from experiments carried out in Xenopus, showing the interaction between the EMI domain and TGF-β3.

1-39. (canceled)

40. A molecule consisting of a cysteine-rich polypeptide sequence defined as “EMI domain”, or sub-fragments thereof, having at least 25% sequence homology to sequence ID NO: 2 which is able to inhibit the conversion of pro-TGF-β to mature TGF-β.

41. The molecule according to claim 40 herein said EMI domain is selected from the group consisting of sequences ID NO: 2, ID NO: 4, ID NO: 6, ID NO: 8, or sub-fragments thereof.

42. The molecule according to claim 41 wherein said sub-fragment is at least 10 amino acids in length.

43. The molecule according to claim 40 comprising chemically modified amino acids and/or uncommon and/or non-natural amino acids or biochemical modifications.

44. The molecule according to claim 43 wherein amino acids are in D- or L-configuration.

45. A pharmaceutical composition comprising as the active principle the molecule according to claim 40 in combination with suitable excipients, diluents and/or delivery systems of the active principle, as a anti-hypertensive.

46. The pharmaceutical composition according to claim 45 for systemic and/or local administration.

47. A method for preparation of a molecule according to claim 40 by recombinant means, wherein a nucleotide sequence encoding a polypeptide with at least 25% sequence homology to SEQ ID NO 2 or a subfragment thereof, is cloned and expressed in a host cell.

48. The method according to claim 47 wherein said nucleotide sequence encodes a molecule selected from the group of: SEQ ID NO 2, SEQ ID NO 4, SEQ ID NO 6, and SEQ ID NO 8.

49. The method according to claim 48 wherein said nucleotide sequences is selected from the group comprising SEQ ID NO, SEQ ID NO 3, SEQ ID NO 5, SEQ ID NO 7 or a sub-fragment thereof.

50. The method according to claim 47 for the preparation of an anti-hypertensive drug.

51. The method according to claim 50 wherein said drug is active in a system selected from the group consisting of: vascular system and/or vascular remodeling and/or atherosclerosis, and/or aneurysms, and/or diabetic vasculopathies.

52. A therapeutic method comprising the administration of a molecule according to claim 40, individually or in combination with other drugs, to a patient affected by a cardiovascular or a vascular pathology selected from the group consisting of: hypertension, diabetic vasculopathies, or atherosclerosis.

53. A method for selecting a biologically active molecule capable of regulating the conversion of pro TGF-β to mature TGF-β, wherein said molecule is placed in contact, at the same or a different time with pro TGF-β and with any one of the molecule according to claim 40 and the conversion of pro-TGF-β into mature TGF-β is measured.

54. The method according to claim 53 for the selection of an anti-hypertensive compound.

55. The method according to claim 53 for the selection of an compound with activity on the vascular system, said activity selected from the group consisting of: vascular remodelling, modification of the vessel lumen diameter, modification of the media layer thickness of a vessel, and smooth muscle cell response to contractile stimuli.


SEQUENCE LISTING (8 sequences)

SEQ ID NO: 1 - DNA, 231 bp, homo sapiens
  CDS (1)..(231): emilin 1 EMI domain aa 55-131
cgccacaggaactggtgtgcctacgtggtgacccggacagtgagctgtgtccttgaggatggagtggagacatatgtcaagtaccagccttgtgcctggggccagccccagtgtccccaaagcatcatgtaccgccgcttcctccgccctcgctaccgtgtggcctacaagacagtgaccgacatggagtggaggtgctgtcagggttatgggggcgatgactgtgctgag

SEQ ID NO: 2 - protein, 77 aa, homo sapiens
RHRNWCAYVVTRTVSCVLEDGVETYVKYQPCAWGQPQCPQSIMYRRFLRPRYRVAYKTVTDMEWRCCQGYGGDDCAE

SEQ ID NO: 3 - DNA, 234 bp, Homo sapiens
  CDS (1)..(234): elastin microfibril interfacer 2 (EMILIN2).EMI domain aa 43-120
aggaacaagaactggtgcgcctacatcgtgaacaagaatgtgagctgctccgtgctggagggaagtgagagttttattcaggctcagtacaactgtgcctggaaccagatgccctgtccgtcggcgctggtgtatcgagtgaacttcagacctagatatgtcactaggtataagacagtgacacagttggaatggaggtgctgtcctggctttagagggggagattgccaagaa

SEQ ID NO: 4 - protein, 78 aa, Homo sapiens
RNKNWCAYIVNKNVSCSVLEGSESFIQAQYNCAWNQMPCPSALVYRVNFRPRYVTRYKTVTQLEWRCCPGFRGGDCQE

SEQ ID NO: 5 - DNA, 234 bp, Homo sapiens
  CDS (1)..(234): multimerin 1 (MMRN1). EMI domain aa 206-283
agaggaaagaattggtgtgcttatgtacataccaggttatctcccacagtgatattggacaaccaggtcacttatgtcccaggtgggaaaggaccttgtggctggaccggtggatcctgtcctcagagatctcagaagatatccaatcctgtctataggatgcaacataaaattgtcacctcattggattggaggtgctgtcctggatacagtgggccgaaatgtcaactaaga

SEQ ID NO: 6 - protein, 78 aa, Homo sapiens
RGKNWCAYVHTRLSPTVILDNQVTYVPGGKGPCGWTGGSCPQRSQKISNPVYRMQHKIVTSLDWRCCPGYSGPKCQLR

SEQ ID NO: 7 - DNA, 234 bp, Homo sapiens
  CDS (1)..(234): multimerin 2 (MMRN2). EMI domain aa 54-131
ggacgtaactggtgcccctacccaatgtccaagctggtcaccttactagctctttgcaaaacagagaaattcctcatccactcgcagcagccgtgtccgcagggagctccagactgccagaaagtcaaagtcatgtaccgcatggcccacaagccagtgtaccaggtcaagcagaaggtgctgacctctttggcctggaggtgctgccctggctacacgggccccaactgcgag

SEQ ID NO: 8 - protein, 78 aa, Homo sapiens
GRNWCPYPMSKLVTLLALCKTEKFLIHSQQPCPQGAPDCQKVKVMYRMAHKPVYQVKQKVLTSLAWRCCPGYTGPNCE